(19) United States
(12) Plant Patent Application Publication
(10) Pub. No.: US 2017/0359938 P1
(43) Pub. Date: Dec. 14, 2017

(54) Petunia Plant Named 'G13460'

(50) ;

(71) Applicant: Michael S. Uchneat, Bellefonte, PA (US)

(72) Inventor: Michael S. Uchneat, Bellefonte, PA (US)

(73) Assignee: GardenGenetics, LLC, Bellefonte, PA (US)

(21) Appl. No.: 14/999,654

(22) Filed: Jun. 9, 2016

(51) Int. Cl. A01H 5/02

(52) U.S. Cl. CPC A01H 5/02
USPC PLT/356.23

(57) ABSTRACT

A new and distinct Petunia plant named ‘G13460’, characterized by its outwardly spreading to trailing and mounding plant habit; vigorous growth habit; freely branching habit; early and freely flowering habit; long flowering period; strong red-colored flowers; and good garden performance.

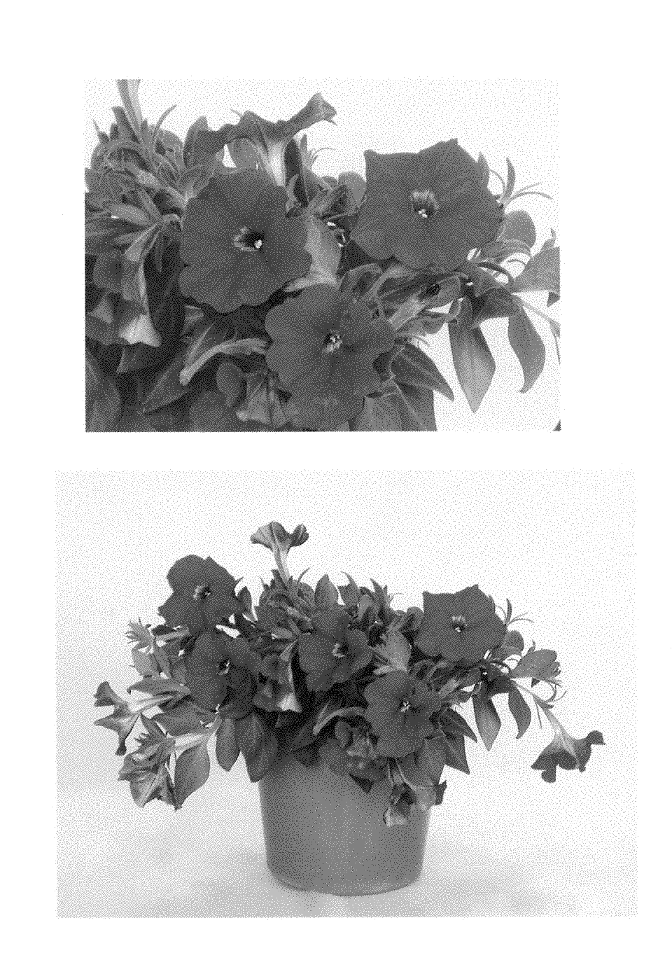

Botanical designation: Petunia×hybrida.

Cultivar denomination: ‘G13460’.

### BACKGROUND OF THE INVENTION

The present invention relates to a new and distinct cultivar of Petunia plant, botanically known as Petunia×hybrida and hereinafter referred to by the name ‘G13460’.

The new Petunia plant is a product of a planned breeding program conducted by the Inventor in Bellefonte, Pa. The objective of the breeding program is to create new freely-branching, cascading to trailing Petunia plants with early and freely flowering habit, unique attractive flowers and good garden performance.

The new Petunia plant originated from a cross-pollination made by the Inventor in January, 2013 in Bellefonte, Pa. of a proprietary seedling selection of Petunia×hybrida identified as code number 8204-1, not patented, as the female, or seed, parent with a proprietary selection of Petunia×hybrida identified as code number 20284-2, not patented, as the male, or pollen, parent. The new Petunia plant was discovered and selected by the Inventor as a single flowering plant within the progeny of the stated cross-pollination in a controlled greenhouse environment in Bellefonte, Pa. on Oct. 18, 2013.

Asexual reproduction of the new Petunia plant by vegetative terminal cuttings in a controlled greenhouse environment in Bellefonte, Pa. since Nov. 13, 2013 has shown that the unique features of this new Petunia plant are stable and reproduced true to type in successive generations.

### SUMMARY OF THE INVENTION

Plants of the new Petunia have not been observed under all possible combinations of environmental conditions and cultural practices. The phenotype may vary somewhat with variations in environmental conditions such as temperature and light intensity without, however, any variance in genotype.

The following traits have been repeatedly observed and are determined to be the unique characteristics of ‘G13460’. These characteristics in combination distinguish ‘G13460’ as a new and distinct Petunia plant:
1. Outwardly spreading to trailing and mounding plant habit.
2. Vigorous growth habit.
3. Freely branching habit.
4. Early and freely flowering habit; long flowering period.
5. Strong red-colored flowers.
6. Good garden performance.

Plants of the new Petunia can be compared to plants of the female parent selection. In side-by-side comparisons, plants of the new Petunia differ primarily from plants of the female parent selection in the following characteristics:
1 Plants of the new Petunia have smaller leaves than plants of the female parent selection.
2. Plants of the new Petunia have smaller flowers than plants of the female parent selection.
3 Throats of flowers of plants of the new Petunia are lighter in color than throats of flowers of plants of the female parent selection.

Plants of the new Petunia can be compared to plants of the male parent selection. In side-by-side comparisons, plants of the new Petunia differ primarily from plants of the male parent selection in the following characteristics:
1. Plants of the new Petunia are more trailing than and not as prostrate as plants of the male parent selection.
2. Plants of the new Petunia are more freely branching than plants of the male parent selection.
3. Plants of the new Petunia have larger flowers than plants of the male parent selection.

Plants of the new Petunia can be compared to plants of ‘USTUNI223’, disclosed in U.S. Plant Pat. No. 14,871. In side-by-side comparisons, plants of the new Petunia differ primarily from plants of ‘USTUNI223’ in the following characteristics:
1. Leaves of plants of the new Petunia are narrower than leaves of plants of ‘USTUNI223’.
2. Plants of the new Petunia and ‘USTUNI223’ differ in flower color as plants of ‘USTUNI223’ have lighter red-colored flowers with darker-colored throat venation.
3. Plants of the new Petunia have longer and more narrow sepals than plants of ‘USTUNI223’.

Plants of the new Petunia can be compared to plants of ‘Balsuncared’, not patented. In side-by-side comparisons, plants of the new Petunia differ primarily from plants of ‘Balsuncared’ in the following characteristics:
1. Plants of the new Petunia are more trailing than and not as upright as plants of ‘Balsuncared’.
2. Plants of the new Petunia and ‘Balsuncared’ differ in flower color as plants of ‘Balsuncared’ have darker red-colored flower throats than plants of the new Petunia.

### BRIEF DESCRIPTION OF THE PHOTOGRAPHS

The accompanying colored photographs illustrate the overall appearance of the new Petunia plant showing the colors as true as it is reasonably possible to obtain in colored reproductions of this type. Colors in the photographs may differ slightly from the color values cited in the detailed botanical description which accurately describe the colors of the new Petunia plant.

The photograph at the bottom of the sheet is a side perspective view of a typical flowering plant of ‘G13460’ grown in a container.

The photograph at the top of the sheet is a close-up view of a typical flowering plant of ‘G13460’.

### DETAILED BOTANICAL DESCRIPTION

The aforementioned photographs and following observations, measurements and values describe plants grown during the autumn in 10-cm containers in an acrylic-covered greenhouse in Carleton, Mich. During the production of the plants, day and night temperatures ranged from 18° C. to 31° C. Plants were six weeks old when the photographs and description were taken. In the following description, color references are made to The Royal Horticultural Society Colour Chart, 2007 Edition, except where general terms of ordinary dictionary significance are used.
Botanical classification: Petunia×hybrida ‘3460’.
Parentage:
   Female, or seed, parent.—Proprietary seedling selection of Petunia×hybrida identified as code number 8204-1, not patented.
   Male, or pollen, parent.—Proprietary seedling selection of Petunia×hybrida identified as code number 20284-2, not patented.
Propagation:
   Type.—Terminal vegetative cuttings.
   Time to initiate roots, summer.—About 7 to 10 days at soil temperatures about 22° C.
   Time to initiate roots, winter.—About 10 to 14 days at soil temperatures about 22° C.
   Time to produce a rooted young plant, summer.—About three to four weeks at ambient temperatures ranging from 22° C. to 27° C.
   Time to produce a rooted young plant, winter.—About four weeks at ambient temperatures ranging from 18° C. to 23° C.
   Root description.—Medium in thickness, fibrous; white in color, actual color of the roots is dependent on substrate composition, water quality, fertilizer type and formulation, substrate temperature and physiological age of roots.
   Rooting habit.—Freely branching; medium density.
Plant description:
   Plant and growth habit.—Outwardly spreading to trailing and mounding plant habit; freely branching habit with about three primary lateral branches each with about five to six secondary lateral branches developing per plant; dense and bushy appearance; vigorous growth habit.
   Plant height, soil level to top of foliar plane.—About 11 cm.
   Plant height, soil level to top of floral plane.—About 11.8 cm.
   Plant diameter (area of spread).—About 29.5 cm.
   Lateral branches.—Length: About 18.5 cm. Diameter: About 3 mm. Internode length: About 2 cm. Strength: Strong. Aspect: Outwardly spreading to trailing. Texture and luster: Pubescent; matte. Color, developing: Close to 145A. Color, developed: Close to 144B.
Leaf description:
   Arrangement.—Alternate before flowering; opposite after flowers develop; leaves simple.
   Length.—About 5.5 cm.
   Width.—About 2.2 cm.
   Shape.—Elliptical.
   Apex.—Acute.
   Base.—Attenuate.
   Margin.—Entire.
   Texture and luster, upper and lower surfaces.—Pubescent; matte.
   Venation pattern.—Pinnate, arcuate.
   Color.—Developing leaves, upper surface: Close to 146B. Developing leaves, lower surface: Close to 146C. Fully expanded leaves, upper surface: Close to 137A; venation, close to 146B. Fully expanded leaves, lower surface: Close to 146B; venation, close to 146D.
   Petioles.—Length: About 1 cm. Diameter: About 3 mm. Strength: Moderately strong. Texture and luster, upper and lower surfaces: Pubescent, matte. Color, upper and lower surfaces: Close to 146D.
Flower description:
   Flower type and flowering habit.—Single axillary salverform flowers; flowers face mostly upward to outwardly; freely flowering habit with about 40 flower buds and open flowers per plant at one time.
   Natural flowering season.—Long day responsive; early flowering habit and long flowering period, plants flower from spring until frost in the autumn, flowering continuous during this period.
   Flower longevity on the plant.—About three days; flowers persistent.
   Fragrance.—None detected.
   Flower buds.—Length: About 4.2 cm. Diameter: About 7 mm. Shape: Oblong, elongate. Texture and luster: Pubescent; slightly glossy. Color: Close to 183D.
   Flower diameter.—About 5.5 cm.
   Flower depth (height).—About 5 cm.
   Throat diameter, distal.—About 1.2 cm.
   Tube length.—About 3.7 cm.
   Tube diameter.—About 6 mm.
   Petals.—Quantity and arrangement: Five petals fused in a single salverform whorl. Petal lobs length (from throat): About 2.2 cm. Petal lobe width: About 2.9 cm. Petal lobe shape: Fan-shaped. Petal lobe apex: Broadly acute. Petal lobe margin: Entire; undulate. Petal lobe texture and luster, upper surface: Smooth, glabrous; velvety, matte. Petal lobe texture and luster, lower surface: Scattered pubescence; matte. Throat texture and luster: Smooth, glabrous; slightly glossy. Tube texture and luster: Pubescent; prominent venation; matte. Color: When opening, upper surface: Close to 53A. When opening, lower surface: Close to 183D. Fully opened, upper surface: Close to 46A to 46B; venation, close to N34A; color does not change with development. Fully opened, lower surface: Close to 181C; venation, close to 182A; color does not change with development. Flower throat (inside): Close to 159A to 159B; venation, close to 199A to 199B. Flower tube (outside): Close to 177B; venation, close to 177A.
   Sepals.—Quantity and arrangement: Five sepals fused in a single star-shaped whorl. Length: About 2.4 cm. Width: About 3 mm. Shape: Lanceolate, Apex: Acute. Margin: Entire. Texture and luster, upper and lower surfaces: Pubescent, minute; matte. Color: When opening, upper surface: Close to 137B. When opening, lower surface: Close to 137C. Fully opened, upper surface: Close to N137B. Fully opened, lower surface: Close to 137C.
   Peduncles.—Length: About 3 cm. Width: About 2 mm. Strength: Strong. Angle: About 35° to 45° from the stem axis. Texture and matte: Pubescent; minute; matte. Color: Close to 144A.
   Reproductive organs.—Stamens: Quantity per flower: Five. Filament length: About 1.4 cm. Filament color: Close to N155C. Anther length: About 1.5 mm. Anther shape: Round. Anther color: Close to 158A. Pollen amount: Moderate. Pollen color: Close to 155C. Pistils: Quantity per flower: One. Pistil length: About 2.5 cm. Style length: About 2.1 cm. Style color: Close to 145D. Stigma diameter: About 1.5 mm. Stigma shape: Round. Stigma color; Close to 144A. Ovary color: Close to 144A.
   Seeds and fruits.—Seed and fruit development has not been observed on plants of the new Petunia.
Pathogen & pest resistance: Plants of the new Petunia have not been noted to be resistant to pathogens or pests common to Petunia plants.
Garden performance: Plants of the new Petunia have been observed to have good garden performance and have been observed to tolerate rain, wind and low temperatures about 2° C. and to be suitable for USDA Hardiness Zone 10.

It is claimed:

1. A new and distinct Petunia plant named ‘G13460’ as illustrated and described.
